(19) United States
(12) Patent Application Publication
(10) Pub. No.: US 2026/0069415 A1
(43) Pub. Date: Mar. 12, 2026

(54) CAPSULE WITH MARKERS FOR ROTATIONAL ALIGNMENT

(71) Applicant: Medtronic, Inc., Minneapolis, MN (US)

(72) Inventors: Jack Francis CULLINAN, Dublin (IE); David FRANCIS, Claremorris MO (IE); Edmond Gerard SHEAHAN, Galway (IE); Mike Francis CONERNEY, Loughrea (IE)

(21) Appl. No.: 19/106,050

(22) PCT Filed: Sep. 13, 2023
(86) PCT No.: PCT/IB2023/059089

§ 371 Date: Feb. 24, 2025

Related U.S. Application Data
(60) Provisional application No. 63/407,105, filed on Sep. 15, 2022.

(51) Int. Cl. A61F 2/24

(52) U.S. Cl. CPC A61F 2/2436; A61F 2250/0098

(57) ABSTRACT

A capsule of a delivery system for a transcatheter heart valve prosthesis includes markers for rotationally orienting the capsule within a native valve. The capsule includes markers that are sized and located on the capsule such that when viewed in a cusp overlap viewing angle image, the markers indicate whether the capsule is in a desired rotational orientation. Methods for rotationally aligning a capsule of a delivery system containing a transcatheter heart valve prosthesis within a native valve are also provided.

### FIELD OF THE INVENTION

The present invention generally relates to medical devices. More particularly, the present technology is related to delivery systems for heart valve prostheses, and in particular to delivery systems including radiopaque markers, and systems and methods for rotationally aligning such delivery systems.

### BACKGROUND

Patients suffering from various medical conditions or diseases may require surgery to install an implantable medical device. For example, valve regurgitation or stenotic calcification of leaflets of a heart valve may be treated with a heart valve replacement procedure. A traditional surgical valve replacement procedure requires a sternotomy and a cardiopulmonary bypass, which creates significant patient trauma and discomfort. Traditional surgical valve procedures may also require extensive recuperation times and may result in life-threatening complications.

One alternative to a traditional surgical valve replacement procedure is delivering implantable medical devices using minimally-invasive techniques. For example, a transcatheter heart valve prosthesis can be percutaneously and transluminally delivered to an implant location. In such methods, the transcatheter heart valve prosthesis can be compressed or crimped on a delivery catheter for insertion within a patient's vasculature; advanced to the implant location; and re-expanded to be deployed at the implant location.

In certain situations, such as, but not limited to, aortic transcatheter heart valve prostheses, it may be desirable to rotationally orient the transcatheter heart valve prosthesis. In delivery systems that radially constrain the transcatheter heart valve prosthesis, such as in a sheath or capsule, it may be beneficial to rotationally orient the transcatheter heart valve prosthesis before the transcatheter heart valve prosthesis is released from the capsule. Thus, a need in the art still generally exists for improved devices and methods for monitoring and tracking the positioning and deployment of the implantable medical device during navigation through or within a patient's anatomy and positioning at the implant site.

### BRIEF SUMMARY OF THE INVENTION

Embodiments hereof are directed to delivery systems including capsules having markers configured to enable a clinician to determine if the capsule is in a desired rotational orientation, and methods of rotationally aligning such capsules.

In a first example, a delivery system includes a capsule disposed at a distal end of the delivery system and configured to radially constrain a heart valve prosthesis in a radially compressed configuration for delivery to a treatment site. A plurality of markers are disposed on the capsule, wherein the plurality of markers are sized, shaped, and located on the capsule such that when viewed in a cusp overlap viewing angle image, the markers indicate whether the capsule is in a desired rotational orientation.

In a second example, in the delivery system of the first example, the desired rotational orientation defines an aligned position or a position within an allowable angle of rotation of the aligned position.

In a third example, in the delivery system of the first example or the second example, the plurality of markers comprise a first marker and a second marker, wherein the first marker and the second marker are spaced 180 degrees apart around a circumference of the capsule.

In a fourth example, in the delivery system of the third example, first marker has a first width and the second marker has a second width, wherein the first width is larger than the second width.

In a fifth example, in the delivery system of the fourth example, the capsule is in the aligned position or the position within an allowable angle of rotation of the aligned position when the second marker is between a right edge and a left edge of the first marker in the cusp overlap viewing angle image.

In a sixth example, in the delivery system of the any one of the third through fifth examples, the first marker and the second marker are longitudinally offset from each other such that one of the first marker and the second marker is closer to a distal end of the capsule than the other of the first marker and the second marker.

In a seventh example, in the delivery system of one of the fourth through sixth examples, the second width is the allowable angle of rotation in radians times the capsule radius plus the first width.

In an eighth example, in the delivery system of the first example or the second example, the plurality of markers comprises a first marker, a second marker, and a third marker spaced 120 degrees apart around a circumference of the capsule, wherein the first marker, second marker, and third marker are configured to be aligned with a commissure of the transcatheter heart valve prosthesis configured to be received in the capsule.

In a ninth example, in the delivery system of the eighth example, the capsule is in the aligned position or the position within an allowable angle of rotation of the aligned position when the first marker and the second marker overlap or are side-by-side with no gap therebetween in the cusp overlap viewing angle image, and the third marker is towards the right of the capsule in the cusp overlap viewing angle image.

In a tenth example, in the delivery system of the ninth example, the first marker and the second marker each have a width about equal to the allowable angle of rotation in radians times a radius of the capsule and divided in half.

In an eleventh example, a method for rotationally aligning a capsule of a delivery system containing a transcatheter heart valve prosthesis within a native heart valve includes percutaneously delivering the capsule with the transcatheter heart valve prosthesis contained therein to the native heart valve, wherein the capsule includes a plurality of markers, obtaining a cusp overlap viewing angle image of the capsule within the native heart valve, determining, based on the cusp overlap viewing angle image and the plurality of markers, whether the capsule is in a desired rotational orientation, if the cusp overlap viewing angle image indicates that the capsule is not in the desired rotational orientation, rotating the capsule until the capsule is in the desired rotational orientation.

In a twelfth example, in the method of the eleventh example, the desired rotational orientation comprises an aligned position or a position within an allowable angle of rotation of the aligned position.

In a thirteenth example, in the method of the eleventh example or the twelfth example, the plurality of markers comprise a first marker and a second marker, wherein the first marker and the second marker are spaced 180 degrees apart around a circumference of the capsule, and a commissure of the transcatheter heart valve prosthesis is spaced 90 degrees from each of the first marker and the second marker.

In a fourteenth example, in the method of the thirteenth example, the first marker has a first width and the second marker has a second width, wherein the first width is larger than the second width.

In a fifteenth example, in the method of the fourteenth example, determining whether the capsule is in a desired rotational orientation comprises determining, based on the cusp overlap viewing angle image, whether the second marker is between a right edge and a left edge of the first marker.

In a sixteenth example, in the method of any one of the thirteenth through fifteenth examples, the first marker and the second marker are longitudinally offset from each other such that one of the first marker and the second marker is closer to a distal end of the capsule than the other of the first marker and the second marker.

In a seventeenth example, in the method of any one of the fourteenth through sixteenth examples, the second width is the allowable angle of rotation in radians times the capsule radius plus the first width.

In an eighteenth example, in the method of the eleventh example or the twelfth example, the plurality of markers comprises a first marker, a second marker, and a third marker spaced 120 degrees apart around a circumference of the capsule, and wherein each of the first marker, second marker, and third marker is rotationally aligned with a corresponding commissure of the transcatheter heart valve prosthesis.

In a nineteenth example, in the method of the eighteenth example, determining whether the capsule is in the desired rotational orientation comprises determining, based on the cusp overlap viewing angle image, whether the first marker and the second marker overlap or are side-by-side with no gap therebetween and whether the third marker is towards the right of the capsule.

In a twentieth example, in the method of the eighteenth example or the nineteenth example, the first marker and the second marker each have a width about equal to the allowable angle of rotation in radians times a radius of the capsule and divided in half.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF DRAWINGS

The foregoing and other features and advantages of the present disclosure will be apparent from the following description of embodiments hereof as illustrated in the accompanying drawings. The accompanying drawings, which are incorporated herein and form a part of the specification, further serve to explain the principles of the present disclosure and to enable a person skilled in the pertinent art to make and use the embodiments of the present disclosure. The drawings may not be to scale.

FIGS. 1A-1B depict illustrations of an example delivery system for a transcatheter heart valve prosthesis.

FIGS. 2A-2C depict illustrations of an example transcatheter heart valve prosthesis that may be used with the delivery systems described herein.

FIG. 3 depicts an illustration of the native aortic valve as viewed from the aorta and depicting various viewing angles for an imaging system.

FIGS. 4A-4B depict an illustration of a capsule of a delivery system, the capsule including imaging markers, according to an embodiment hereof.

FIG. 5A depicts an illustration of a native aortic valve as viewed from the aorta and including the capsule of FIG. 4A showing an aligned position, according to an embodiment hereof.

FIG. 5B depicts a schematic representation of a fluoroscopic image of a native aortic valve using the cusp overlap view and showing the markers of the capsule FIG. 4A with the capsule in an aligned position.

FIG. 6A depicts an illustration of a native aortic valve as viewed from the aorta and including the capsule of FIG. 4A showing the capsule rotated 20° from the aligned position in a clockwise direction, according to an embodiment hereof.

FIG. 6B depicts a schematic representation of a fluoroscopic image of a native aortic valve using the cusp overlap view and showing the markers of the capsule FIG. 4A with the capsule in the position shown in FIG. 6A.

FIG. 7A depicts an illustration of a native aortic valve as viewed from the aorta and including the capsule of FIG. 4A showing the capsule rotated 20° from the aligned position in a counter-clockwise direction, according to an embodiment hereof.

FIG. 7B depicts a schematic representation of a fluoroscopic image of a native aortic valve using the cusp overlap view and showing the markers of the capsule FIG. 4A with the capsule in the position shown in FIG. 7A.

FIG. 8A depicts an illustration of a native aortic valve as viewed from the aorta and including the capsule of FIG. 4A showing the capsule rotated 45° from the aligned position in a clockwise direction, according to an embodiment hereof.

FIG. 8B depicts a schematic representation of a fluoroscopic image of a native aortic valve using the cusp overlap view and showing the markers of the capsule FIG. 4A with the capsule in the position shown in FIG. 8A.

FIGS. 9A-9B depict an illustration of a second embodiment of markers on a capsule of a delivery system, according to an embodiment hereof.

FIG. 10A depicts an illustration of a native aortic valve as viewed from the aorta and including the capsule of FIG. 9A showing an aligned position, according to an embodiment hereof.

FIG. 10B depicts a schematic representation of a fluoroscopic image of a native aortic valve using the cusp overlap view and showing the markers of the capsule FIG. 9A with the capsule in the position shown in FIG. 10A.

FIG. 11A depicts an illustration of a native aortic valve as viewed from the aorta and including the capsule of FIG. 9A showing the capsule rotated 20° from the aligned position in a clockwise direction, according to an embodiment hereof.

FIG. 11B depicts a schematic representation of a fluoroscopic image of a native aortic valve using the cusp overlap view and showing the markers of the capsule FIG. 9A with the capsule in the position shown in FIG. 11A.

FIG. 12A depicts an illustration of a native aortic valve as viewed from the aorta and including the capsule of FIG. 9A showing the capsule rotated 20° from the aligned position in a counter-clockwise direction, according to an embodiment hereof.

FIG. 12B depicts a schematic representation of a fluoroscopic image of a native aortic valve using the cusp overlap view and showing the markers of the capsule FIG. 9A with the capsule in the position shown in FIG. 12A.

FIG. 13A depicts an illustration of a native aortic valve as viewed from the aorta and including the capsule of FIG. 9A showing the capsule rotated 60° from the aligned position in a clockwise direction, according to an embodiment hereof.

FIG. 13B depicts a schematic representation of a fluoroscopic image of a native aortic valve using the cusp overlap view and showing the markers of the capsule FIG. 9A with the capsule in the position shown in FIG. 13A.

### DETAILED DESCRIPTION

It should be understood that various embodiments disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single device or component for purposes of clarity, it should be understood that the techniques of this disclosure may be performed by a combination of devices or components associated with, for example, a delivery device. The following detailed description is merely exemplary in nature and is not intended to limit the invention of the application and uses of the invention. Furthermore, there is no intention to be bound by any expressed or implied theory presented in the preceding field of the invention, background, summary, or the following detailed description.

As used in this specification, the singular forms “a,” “an,” and “the” specifically also encompass the plural forms of the terms to which they refer, unless the content clearly dictates otherwise. The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 5%. It should be understood that use of the term “about”also includes the specifically recited number or value.

The terms “proximal” and “distal” herein when used with respect to a delivery system are used with reference to the clinician using the devices. Therefore, “proximal” and “proximally” mean in the direction toward the clinician, and “distal” and “distally” mean in the direction away from the clinician. The terms “proximal” and “distal” herein when used with respect to a medical device to be implanted, such as a heart valve prosthesis, are used with reference to the direction of blood flow. Therefore, “proximal” and “proximally” mean in an upstream direction, and “distal”and “distally”mean in a downstream direction.

As used herein, the term “generally” and “substantially” mean approximately. When used to describe angles such as “substantially parallel” or “substantially perpendicular,” the term “substantially” means within 10 degrees of the angle. When used to describe shapes such as “substantially” or “generally” cylindrical, “substantially” or “generally” tube-shaped, or “substantially” or “generally” conical, the terms mean that the shape would respectively appear cylindrical, tube-shaped, or conical to a person of ordinary skill in the art viewing the shape with a naked eye.

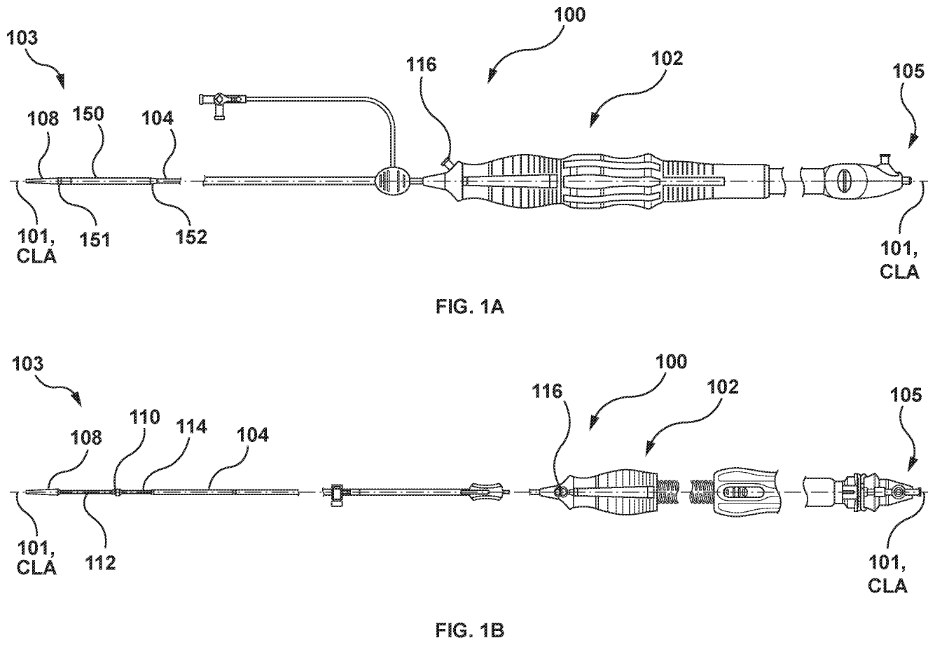

FIGS. 1A-1B illustrate an example delivery system 100 for a transcatheter heart valve prosthesis, such as the transcatheter heart valve prosthesis 200 that will be discussed in further detail below. In an embodiment, the transcatheter heart valve prosthesis is self-expanding. The delivery system 100 includes a distal end 103 and a proximal end 105.

In particular, the delivery system 100 includes a handle 102. The handle 102 enables a clinician to manipulate a distal end 103 of the delivery system 100 and includes actuators for moving parts of the delivery system 100 relative to other parts. In the delivery system 100, an outer shaft 104 is coupled to an actuator of the handle 102 for moving the outer shaft 104 relative to an inner shaft 112. A distal portion of the outer shaft 104, referred to as a capsule 150, is configured to surround the transcatheter heart valve prosthesis 200 during delivery to the treatment site, e.g., a native heart valve and is retracted from the transcatheter heart valve prosthesis 200 to expose the transcatheter heart valve prosthesis 200 such that it self-expands. The inner shaft 112 is coupled to the handle 102 such that movement of the handle translates to movement of the inner shaft 112 and a distal tip 108 coupled to a distal end of the inner shaft 112. The inner shaft 112 and distal tip 108 may also be translated relative to the outer shaft 104 and the handle 102 via a tip retractor. In the embodiment shown, a middle member 114 is disposed between the inner shaft 112 and the outer shaft 104, and the middle member 114 includes a retainer or spindle 110 attached to a distal portion thereof for receiving paddles 250 of the transcatheter heart valve prosthesis 200, which is described in further detail below.

A flush port 116 is disposed on the handle 102. In the delivery system 100 shown, when the transcatheter heart valve prosthesis 200 is properly loaded into the delivery system 100, certain relationships between features of the transcatheter heart valve prosthesis 200 and features of the delivery system 100 are present, which may assist in predicting the proper rotational orientation of the transcatheter heart valve prosthesis 200 disposed within the capsule 150. In particular, when loading the transcatheter heart valve prosthesis 200 into the delivery system 100, the paddles 250 are placed into paddle pockets (not shown) of the spindle 110 at 180° apart from each other. As described in further detail below, the paddle 250 with the C-shaped marker (sometimes referred to as a “C-tab” or “C-paddle”) is aligned with a commissure 209 of the transcatheter heart valve prosthesis 200. Further, when the transcatheter heart valve prosthesis 200 is loaded into the delivery system 100, the C-paddle 250 is located in the paddle pocket that is aligned with the flush port 116 on the handle 102 of the delivery system 100.

As noted above, this is a brief description of an example delivery system 100. Other parts shown in FIGS. 1A-1B are not described in detail herein and would be apparent to those skilled in the art.

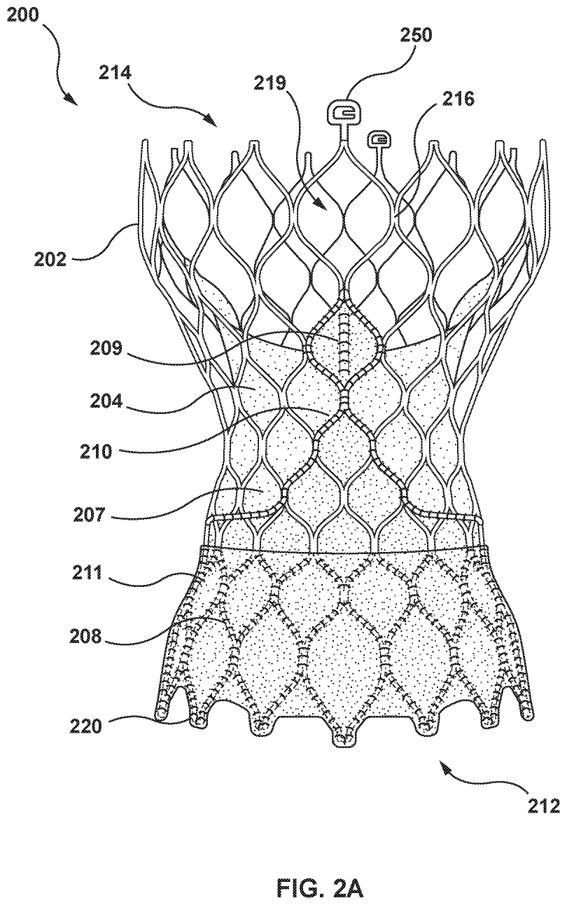

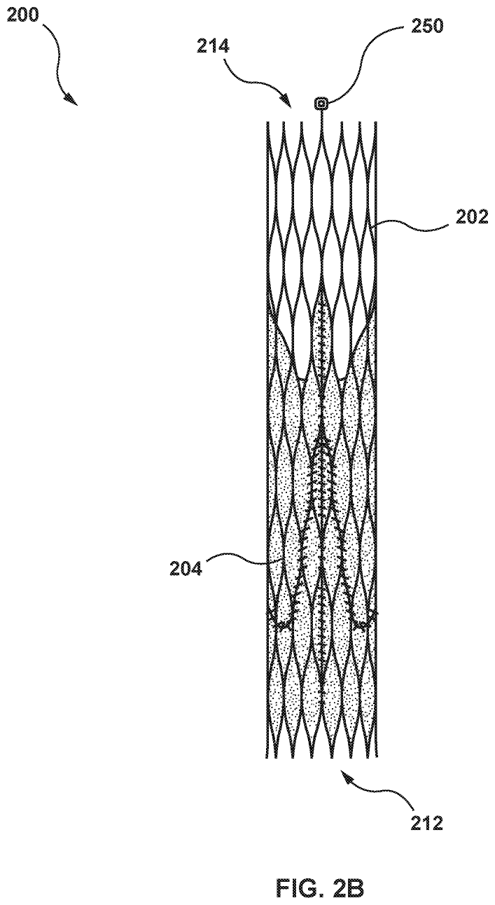

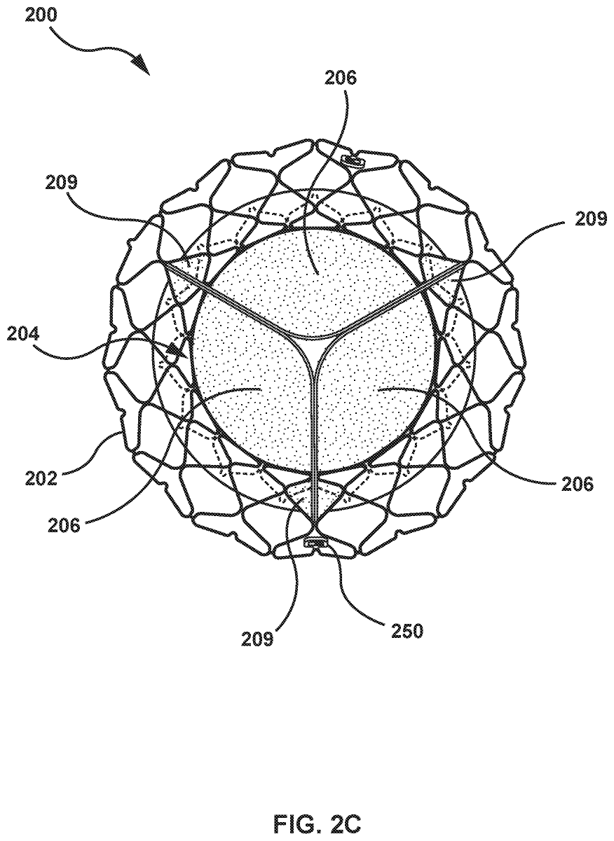

FIGS. 2A-2C illustrate an example of a transcatheter heart valve prosthesis 200. Although details of the heart valve prosthesis 200 are described herein, they are not meant to be limiting, and the systems and methods described herein can be used with any transcatheter heart valve prosthesis. FIG. 2A is side view of a transcatheter heart valve prosthesis 200 in a normal or expanded (uncompressed) configuration. FIG. 2B illustrates the transcatheter heart valve prosthesis 200 in a compressed configuration (e.g., when compressively retained within a delivery system such as a distal portion of a delivery system, as known to those skilled in the art). The transcatheter heart valve prosthesis 200 includes a stent or frame 202 (hereinafter “stent”) and a valve structure 204. The stent 202 can assume any of the forms described herein and variations thereof, and is generally constructed so as to be expandable from the compressed configuration (FIG. 2B) to the uncompressed, normal, or expanded configuration (FIG. 2A). In some embodiments, the stent 202 is self-expanding. The valve structure 204 is assembled to the stent 202 and provides two or more (typically three) leaflets 206, as illustrated in further detail below with reference to FIG. 2C.

In embodiments, the valve structure 204 can be coupled to the stent 202 in various manners, such as by sewing the valve structure 204 to one or more struts 208 or commissure posts defined by the stent 202 using sutures 210. The valve structure 204 is capable of blocking flow in one direction to regulate flow there-through via valve leaflets 206 that may form a bicuspid or tricuspid replacement valve. The valve leaflets 206 may be attached to an interior skirt or graft material 207 which encloses or lines a portion of the stent 202 as would be known to one of ordinary skill in the art of prosthetic tissue valve construction. The valve leaflets 206 may be sutured or otherwise securely and sealingly attached along their bases with the sutures 210 to the interior surface of the interior skirt 207. The transcatheter heart valve prosthesis 200 can also include an exterior skirt (not shown) coupled to the outer surface of the stent 202 at the inflow end 212 thereof. The exterior skirt may be attached to stent 202 by any suitable means known to those skilled in the art, for example and not by way of limitation, suture/stitches, welding, adhesive, or other mechanical coupling. Adjoining pairs of leaflets are attached to one another at their lateral ends to form commissures 209, with the free edges of the leaflets forming coaptation edges that meet in an area of the coaptation. In the embodiment shown, the commissures 209 are configured to span a cell of the stent 202, so that force is evenly distributed within the commissures and the stent 202, however, this is not meant to be limiting.

The transcatheter heart valve prosthesis 200 of FIGS. 2A-2C can be configured to replace or repair an aortic valve. Alternatively, other shapes are also envisioned, adapted to the specific anatomy of the valve to be repaired (e.g., stented prosthetic heart valves in accordance with the present disclosure can be shaped and/or sized for replacing a native mitral, pulmonic, or tricuspid valve). With the example of FIG. 2A, the valve structure 204 extends less than the entire length of the stent 202, but in other embodiments can extend along an entirety, or a near entirety, of a length of the stent 202. A wide variety of other constructions are also acceptable and within the scope of the present disclosure. For example, the stent 202 can have a more cylindrical shape in the normal, expanded arrangement.

The stent 202 includes struts 208 that operate as support structures arranged relative to each other to provide a desired compressibility and strength to the transcatheter heart valve prosthesis 200. For example, as best illustrated in FIG. 2A, the struts 208 are arranged in such a manner as to form windows or cells 219 around the circumference and along the length of the stent 202. Longitudinally adjacent rows of struts 208 and crowns 220 are joined together at the crowns 220, thereby forming nodes 216. The arrangement of struts 208 and crowns 220 forming rows of cells 219 forms a central lumen or passageway and from the inflow end 212 and an outflow end 214. As illustrated, in embodiments, the overall structure formed by the struts 208 and crowns 220 can form the stent 202 having a generally hourglass shape in which the inflow end 212 and the outflow end 214 have a diameter that is larger than a middle portion of the stent 202. However, this shape is not meant to be limiting and other shapes, such as more of a cylindrical shape, may be utilized. The stent 202 can be formed by a laser-cut manufacturing method and/or another conventional stent forming method as would be understood by one of ordinary skill in the art. A lateral cross-section of the stent 202 can be trapezoidal, circular, ellipsoidal, rectangular, hexagonal, square, or other polygonal shape, although at present it is believed that trapezoidal, circular or ellipsoidal may be preferable when utilized with the replacement of an aortic valve.

In embodiments, as illustrated in FIG. 2A the stent 202 can also include one or more paddles 250 that removably couple the prosthetic heart valve 200 to a delivery system, such as the delivery system 100. For example, and not by way of limitation, a delivery system known as the En Veo™ PRO catheter or the En Veo™ R catheter, from Medtronic, Inc. may be utilized. While FIGS. 2A and 2C illustrate two (2) paddles 250, one skilled in the art will realize that the paddles 250 can be replaced with other components such as eyelets, loops, slots, or any other suitable coupling member, and that more or fewer paddles or other coupling members may be utilized. In the embodiment shown, at least one of the paddles 250 is radiopaque so as to be visible under fluoroscopy, with the radiopaque paddle in this embodiment including a C-shaped marker to assist with orientation of the transcatheter heart valve prosthesis 200 during implantation. Those skilled in the art would recognize that other asymmetric shapes may be utilized so assist in determining the orientation of the transcatheter heart valve prosthesis 200 during implantation. In embodiments, such as the embodiment of FIGS. 2A-2C, the paddle 250 with the C-shaped marker is axially aligned with one of the commissures 209 of the valve structure 204, as best seen in FIG. 2A.

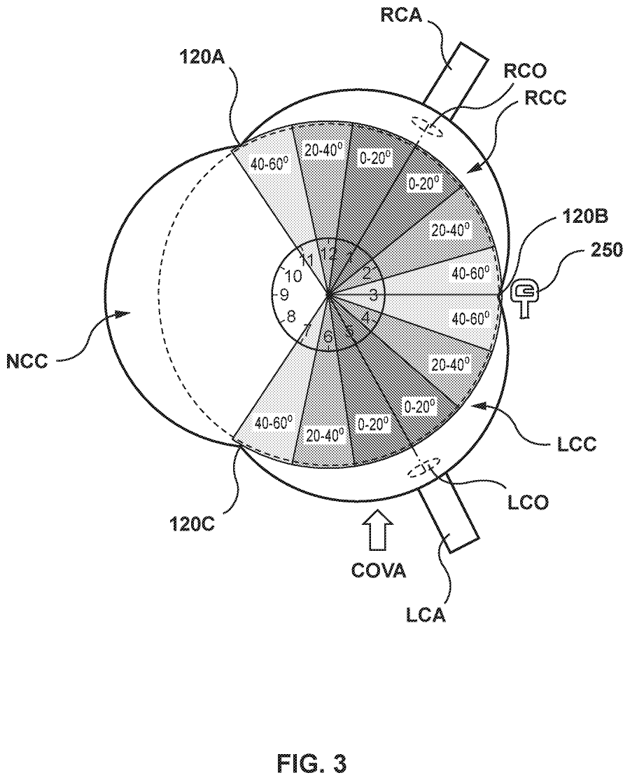

Referring now to FIG. 3, a schematic illustration of an exemplary native aortic valve in a view from the aorta is shown. As can be seen, the native aortic valve includes three leaflets or cusps, a right coronary cusp RCC, a left coronary cusp LCC, and a non-coronary cusp NCC. As known to those skilled in the art, the right coronary artery includes an ostium or opening RCO in the sinus of Valsalva, superior to the right coronary cusp RCC and inferior to the sinotubular junction (not shown). Similarly, the left coronary artery includes an ostium or opening LCO in the sinus of Valsalva, superior to the left coronary cusp RCC and inferior to the sinotubular junction (not shown). Further, the non-coronary cusp NCC is in the sinus that does not include an ostium or opening for a coronary artery.

As known to those skilled in the art, and shown in FIG. 3, the leaflets or cusps are joined at commissures. Thus, the right-non-coronary commissure 120A is where the right coronary cusp RCC and the non-coronary cusp NCC are joined, the left-right commissure 120B is where the left coronary cusp LCC and the right coronary cusp RCC are joined, and the left-non-coronary commissure 120C is where the left coronary cusp LCC and the non-coronary cusp NCC are joined. FIG. 3 shows an idealized native aortic valve with the left coronary ostium LCO and the right coronary ostium spaced 120 degrees apart. However, as known to those skilled in the art, there may be patient specific variation of 10-20 degrees in the location of the commissures. Further, it is noted that the commissures are not exactly 120 degrees apart. Instead, on average, the left-right commissure LRC is closer to the left-non-coronary commissure LNC, at approximately 108 degrees, than to the other two commissures. Further, the location of the ostia of the left and right coronary arteries may vary approximately 15-20 degrees depending on patient anatomy. In FIG. 3, a clock face has been added and is centered within the exemplary native aortic heart valve and used herein to aid in referencing various positions within the native aorta. For example, FIG. 3 shows the right coronary ostium RCO at 1 o'clock and the left coronary ostium LCO at 5 o'clock with regard to the clock face. Similarly, the native commissures 120A, 120B and 120C are shown at 11 o'clock, 3 o'clock, and 7 o'clock, respectively.

With this understanding, imaging systems such as fluoroscopic imaging systems used during transcatheter heart valve replacement procedures generally include a C-arm gantry that enables different viewing angles of the native aortic valve. One particular viewing angle is the cusp overlap viewing angle COVA. In the cusp overlap view, as shown in FIG. 3, the viewing angle of the imaging system is such that the right coronary cusp RCC and the left coronary cusp LCC overlap each other. In the cusp overlap view, the non-coronary cusp NCC is to the left of the left coronary cusp LCC and the right coronary cusp RCC. Further in the cusp overlap viewing angle, the right-non-coronary commissure 120A and the left-non-coronary commissure 120C are aligned with each other, i.e., they overlap. In the cusp overlap view, the left-right commissure 120B is to the right of the right-non-coronary commissure 120A and the left-non-coronary commissure 120C. FIG. 3 shows the cusp overlap viewing angle COVA as indicated by the arrow.

FIG. 3 also shows the location of the C-paddle 250 of the transcatheter heart valve prosthesis 200. As described above, the C-paddle 250 is axially aligned with a commissure 209 of the valve structure 204. It is desirable for the commissures 209 of the valve structure 204 of the transcatheter heart valve prosthesis 200 to be aligned with the native commissures 120A, 120B, 120C of the native aortic valve. In the idealized native aortic valve shown in FIG. 3, the native commissures are shown 120 degrees apart from each other. As described above with respect to the transcatheter heart valve prosthesis, the commissures 209 of the valve structure 204 are 120 degrees apart from each. Thus, the commissures 209 may be aligned with the native commissures 120. Aligning the prosthetic commissures with the native commissures provides many benefits including, but not limited to, not blocking the coronary ostia RCO, LCO. Those skilled in the art would recognize that in practice, the native anatomy is generally not the idealized anatomy described herein. Nevertheless, the principles described herein can be used and adapted to the particular anatomy of a patient, as determined by a pre-procedure CT scan.

Referring to FIG. 3, the C-paddle 250, and hence one of the commissures 209 of the valve structure 204 is aligned with the right-left commissure 120B. In an embodiment, it is preferred that the commissure 209 of the valve structure 204 be within 20 degrees of right-left commissure 120B. This is shown in FIG. 3 as C-paddle 250 being in the zone 40°-60° (40-60 degrees) from the right coronary ostium RCO and 40°-60° (40-60 degrees) from the left coronary ostium LCO. Thus, this provides a 40 degree rotational range for the transcatheter heart valve prosthesis 200 to be oriented with respect to the native valve and still be considered acceptable. Those skilled in the art will recognize that the ranges noted above are not meant to be limiting, and the systems and techniques described below can be modified and used with other ranges that may be considered acceptable, either smaller or larger ranges.

Having generally described the delivery system 100 and the transcatheter heart valve prosthesis 200, particular embodiments of the capsule 150 with imaging markers, and methods and systems for using the capsule 150 with imaging markers to rotationally orient the transcatheter heart valve prosthesis 200, will be described.

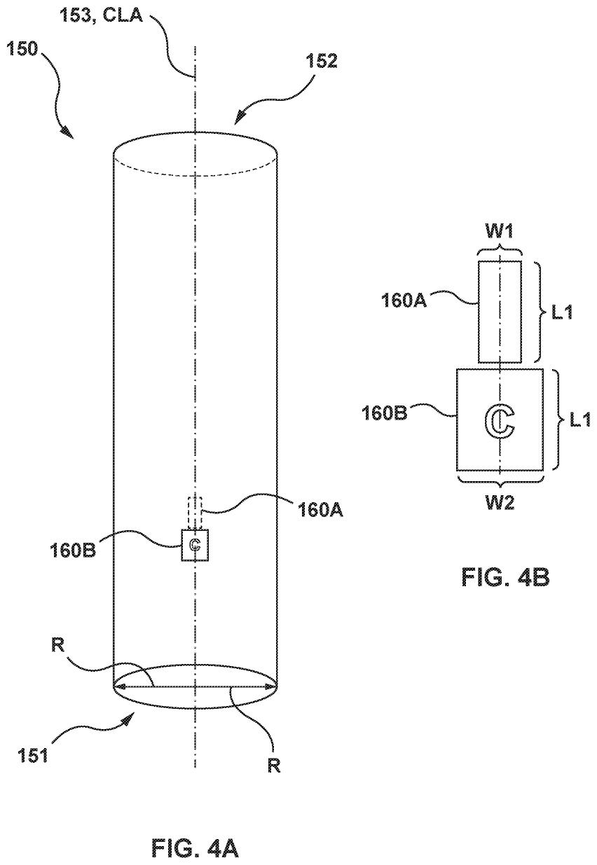

FIGS. 4A-4B show an embodiment of imaging markers disposed on the capsule 150 of the delivery system 100. FIG. 4A shows a close up view of the capsule 150 of the delivery system 100. As can be seen, the capsule 150 of the delivery system 100 is a tubular structure including a distal end 151 and a proximal end 152. An interior surface of the capsule 150 defines a central lumen 153 that runs parallel to the central longitudinal axis CLA of the delivery system 100 and extends an entire longitudinal length of the capsule 150, extending from the distal end 151 to the proximal end 152. In an embodiment, the capsule 150 has a radius R of about 2.5-3.0 mm. However, this is not meant to be limiting, and the capsule 150 may have a radius (and diameter) as required to radially constrain a given transcatheter heart valve prosthesis. Thus, for transcatheter heart valve prostheses with a larger diameter when radially compressed, the capsule may have a larger diameter, and for transcatheter heart valve prostheses with a smaller diameter when radially compressed, the capsule may have a smaller diameter. The capsule 150, disposed at the distal end 103 of the delivery system 100, constrains the transcatheter heart valve prosthesis 200 in the radially compressed configuration, shown in FIG. 2B, during delivery to the treatment site. When the delivery system 100 is disposed at the treatment site, in this example a native aortic valve, the capsule 150 is withdrawn proximally (in FIG. 4A upwardly) to expose the self-expanding transcatheter heart valve prosthesis 200. Thus, in the embodiment shown, the inflow end 212 of the transcatheter heart valve prosthesis 200 is exposed first.

As shown in FIG. 4A, the capsule 150 includes imaging markers 160 disposed on the capsule 150. More specifically, the imaging markers 160 can be attached to, positioned in, and/or formed in the capsule 150 utilizing any type of processes and/or procedure. Further, for example, and not by way of limitation, the markers 160 may be press-fit, electroplated, printed on, or otherwise attached or added to the capsule 150. In any embodiment, the imaging markers 160 may include radiopaque or other material that allows the marker 160 to be detected and/or viewed under radiography during the implantation of the transcatheter heart valve prosthesis 200 via the delivery system 100. Examples of radiopaque materials include metals, e.g., platinum-iridium, gold, iridium, palladium, rhodium, titanium, tantalum, tungsten and alloys thereof. Other examples of radiopaque material include polymeric materials, e.g., nylon, polyurethane, silicone, PEBAX, PET, polyethylene, that have been mixed or compounded with compounds of barium, bismuth and/or zirconium, e.g., barium sulfate, zirconium oxide, bismuth sub-carbonate, etc. In embodiments, gold is a preferred marker material due to its visibility. Further, in addition to the radiopaque materials noted above, the imaging markers 160 can be a feature on the capsule 150 of the delivery system 100 that can be seen under fluoroscopy as distinguished from other features of the delivery system 100.

In the embodiment of FIGS. 4A-4B, the capsule 150 includes two markers 160A, 160B. As shown in FIG. 4A, the markers 160A and 160B are disposed 180 degrees apart from each other. In other words, in the embodiment of FIG. 4A, the marker 160B is in the “front” of the capsule 150 and the marker 160A is in the “back” of the capsule 150 when the capsule 150 is viewed from the front. Those skilled in the art will realize that “front” and “back” are used with reference only to FIG. 4A to refer to the relative positions of the markers 160A, 160B, and are not meant to be limiting.

Further, in the embodiment of FIGS. 4A-4B, the markers 160A, 160B are longitudinally offset from each other. In other words, one of the markers is distal of the other marker. In the embodiment of FIGS. 4A-4B, the marker 160B is distal of the marker 160A. In other words, the marker 160B is closer to the distal end 151 of the capsule 150 than the marker 160A. Thus, in the embodiment shown, the marker 160A is disposed proximal to marker 160B, and marker 160B is disposed distal to marker 160A. However, this is not meant to be limiting, and either of the markers may be the distal marker and/or the proximal marker. In the embodiment shown, the markers 160A, 160B are longitudinally offset such that they will not obscure each other when viewed in a fluoroscopic image, as described in more detail below.

FIG. 4B shows a close up view of markers 160A and 160B. In the embodiment of FIGS. 4A-4B, each of the markers 160A and 160B are rectangular in shape. However, this is not meant to be limiting, and other shapes may be used. The markers 160A and 160B are sized such that when viewed in a fluoroscopic image in a cusp overlap viewing angle, the position of the marker 160A relative to the marker 160B enables a clinician to determine whether the transcatheter heart valve prosthesis 200 is rotationally oriented such as to avoid blocking the ostia of the coronary arteries, as described in more detail below. Thus, the markers 160A, 160B are sized relative to each other to provide a “tolerance” of positions relative to each other when viewed in a fluoroscopic image, as described in more detail below. In the embodiment of FIGS. 4A-4B, and as described in more detail below, when the marker 160A is within the width of the marker 160B in the cusp overlap view, the capsule 150 is rotationally oriented such that the commissure 209 of the heart valve prosthesis 200 is within the 40 degrees allowable angle of rotation θ (Theta) tolerance angle from the native right-left commissure 120B. Thus, in an embodiment with a capsule diameter of about 5.56 mm (radius R of about 2.78 mm), and given the allowable angle of rotation θ (Theta) or tolerance angle described above of 40 degrees (about 0.7 radians) (20 degrees in either direction), with the marker 160A having a width W1 of about 1 mm, the marker 160B will have a width W2 of about 2.94 mm to function as described below (W2=Allowable Angle of Rotation (about 0.7 radian)×capsule radius (2.78 mm)+W1=2.946 mm). Further, in the embodiment shown, the marker 160B includes an asymmetrical shape, in this example a C-shape, to assist in determining whether the marker 160B is anterior or posterior in the fluoroscopic image with the gantry in the cusp overlap viewing angle (also referred to as the “cusp overlap viewing angle image”). Those skilled in the art would recognize that other asymmetric shapes may be utilized to assist in determining the orientation of the capsule 150 during implantation.

In the embodiment shown, each of the markers 160 are positioned on the exterior surface of the capsule 150 with a known radial relation to the C-paddle 250 on the transcatheter heart valve prosthesis 200. The C-paddle 250 of the transcatheter heart valve prosthesis 200 is axially aligned with one of the three commissures 209 of the valve structure 204. Thus, knowing the radial relation of the markers 160 on the capsule 150 in relation to the C-paddle 250 of the transcatheter heart valve prosthesis 200 enables a user to rotationally align the transcatheter heart valve prosthesis 200 in situ such that the commissures 209 of the transcatheter heart valve prosthesis 200 do not block access to the left and right coronary ostia LCO, RCO of the native heart valve when the transcatheter heart valve prosthesis 200 is deployed, because the user can determine where the commissures 209 of the transcatheter heart valve prosthesis 200 are located based on the markers 160 of the capsule 150, which will be described in further detail below. However, although described with respect to the radial relation between markers 160 and the C-paddle 250, this is not meant to be limiting, and other features of a transcatheter heart valve prosthesis and its orientation when loaded into a capsule may be utilized.

With the above understanding of the cusp overlap view, a system and method for rotationally aligning a capsule 150 of a delivery system 100, containing a compressed transcatheter aortic valve prosthesis 200, will now be described. The system and method described is with respect to the capsule 150 of the delivery system 100 described above, as well as the transcatheter heart valve prosthesis 200 described above, and markers 160A and 160B described above. However, it would be understood by those skilled in the art that the system and method described may be utilized with other capsules, delivery systems, and/or transcatheter heart valve prostheses with more or few markers and disposed in different locations. Specific variations will be discussed in more detail below, but are also not intended to be limiting.

In particular, the desired rotational alignment of the capsule 150, and therefore the implanted transcatheter heart valve prosthesis 200, is to ensure that the commissures 209 of the transcatheter heart valve prosthesis 200 do not block access to the left and right coronary ostia RCO, LCO. In particular, after implantation of the transcatheter heart valve prosthesis 200, it may be necessary for an interventional treatment within one of the patient's coronary arteries, such as angioplasty or stent implantation, for example. However, if one of the prosthetic valve commissures or prosthetic tissue adjacent thereto is blocking the coronary artery, a clinician may not be able to access the coronary artery for the post-implantation procedure. In the embodiments described in more detail below, systems and methods for rotationally aligning a prosthetic valve commissure (such as one of the commissures 209 of the transcatheter heart valve prosthesis 200) with respect to one of the native valve commissures may be sufficient to ensure coronary access. Precise prosthetic valve/native valve commissure alignment is not required, as the goal is coronary access. Other benefits from substantial commissure alignment include improved valve durability and resistance to thrombogenicity, and potential alignment of a second transcatheter heart valve prosthesis in a valve-in-valve procedure. With transcatheter aortic valve replacement procedures being performed using 2-dimensional imaging, such as fluoroscopy, the rotational position of prosthetic valve commissures with respect to the native valve commissures or coronary ostia is difficult to determine.

With the above understanding of the markers 160 on the capsule 150 of the delivery system 100, a system and method of rotationally aligning the capsule 150 of the delivery system 100, and therefore the transcatheter heart valve prosthesis 200, will now be described. As known to those skilled in the art, the transcatheter heart valve prosthesis 200 may be delivered percutaneously via femoral access. In particular, in the example of a self-expanding transcatheter heart valve prosthesis, e.g. the transcatheter heart valve prosthesis 200, the prosthesis is constrained in a radially compressed configuration by, for example, the capsule 150 of the delivery system 100. Characteristics of a patient's native anatomy may be determined prior to the procedure, such as by a CT scan. In particular, the coronary ostia (LCO, RCO) may be located using a CT scan. Using this planning CT, a determination may be made prior to the procedure regarding orientation of the delivery system, and hence the transcatheter heart valve prosthesis, when delivering the transcatheter heart valve prosthesis. For example, and not by way of limitation, the delivery system 100 can be arranged such that the flush port 116 is aligned with the C-paddle 250 of the transcatheter heart valve prosthesis 200, which is aligned with one of the commissures 209 of the valve structure 204. Using pre-procedure CT, the orientation of a feature of the delivery system, such as the flush portion 116, that has a known relationship to a feature of the transcatheter heart valve prosthesis, such as one of the commissures 209 of the transcatheter heart valve prosthesis 200, may be further defined by the specific patient anatomy. Thus, using pre-procedure planning, a prediction can be made regarding a preferred orientation of the delivery system, such as the delivery system 100, to reduce coronary artery ostia overlap.

Further, during the procedure, the coronary overlap view and the markers 160 may be used to confirm that the transcatheter heart valve prosthesis is rotationally aligned such as to not cause coronary obstruction. The capsule 150 may then be retracted proximally to expose the transcatheter heart valve prosthesis 200, enabling the transcatheter heart valve prosthesis 200 to self-expand.

Thus, when the delivery system 100 is advanced to the site of a native heart valve, for example a native aortic valve, the markers 160A, 160B can be used to confirm that the capsule 150 is in a desired rotational orientation within the native valve. In particular, the cusp overlap viewing angle discussed above may be utilized to determine if the capsule 150, and hence the transcatheter heart valve prosthesis 200 disposed therein, are properly rotationally oriented.

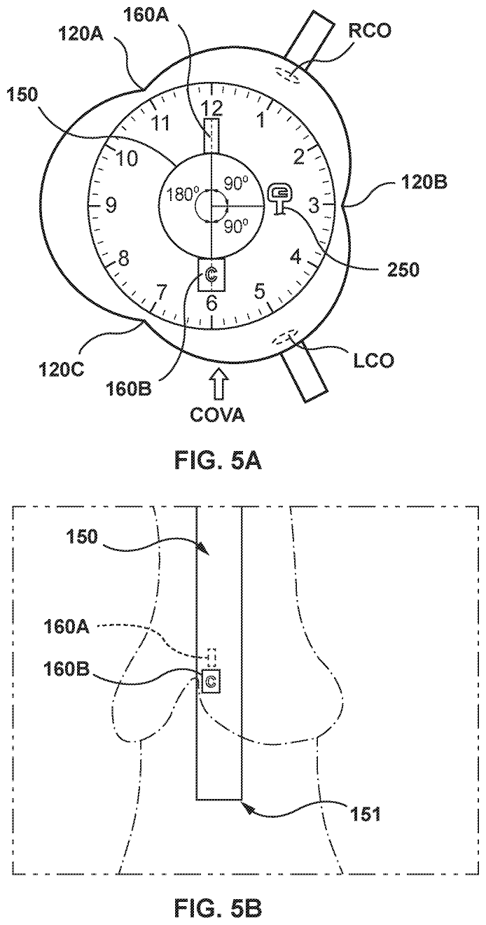

FIGS. 5A-5B depict the markers 160A and 160B on the capsule 150 of the delivery system 100, as introduced in FIGS. 4A-4B, in a specific rotational position termed an “aligned position”, according to embodiments hereof. The “aligned position” as used herein is with respect to the capsule's 150 rotational orientation with respect to the native aortic valve, and hence the position of the transcatheter heart valve prosthesis 200 disposed within the capsule 150 with respect to the native aortic valve.

As stated previously, markers 160A and 160B are disposed 180 degrees from one another on the capsule 150. The transcatheter heart valve prosthesis 200 is disposed within the central lumen 153 of the capsule 150 in the compressed configuration. In the embodiment described, when the transcatheter heart valve prosthesis 200 is loaded into the capsule 150 in the compressed configuration, the C-paddle 250 of the transcatheter heart valve prosthesis 200 is disposed 90 degrees from both markers 160A and 160B, as best shown in FIG. 5A. FIG. 5A shows the capsule 150 of the delivery system 100 within an exemplary native aortic valve as viewed from the aorta. The capsule 150 includes the markers 160A, 160B from FIGS. 4A-4B. As can be seen, marker 160A and marker 160B are disposed 180 degrees apart from one another on the capsule 150. With reference to the clock face, marker 160A is positioned at 12 o'clock and marker 160B is positioned at 6 o'clock. As explained above, the transcatheter heart valve prosthesis 200 is disposed within the central lumen 153 of the capsule 150 in the compressed configuration for delivery. The C-paddle 250 of the transcatheter heart valve prosthesis 200 is aligned with one of the commissures 209 of the transcatheter heart valve prosthesis 200. As shown in FIG. 5A, the C-paddle 250 is positioned at 3 o'clock within the native aortic valve. As shown in FIG. 5A, markers 160A and 160B are both disposed 90 degrees from the C-paddle 250 in opposite directions from the C-paddle 250. In other words, the markers 160 of the capsule 150 are equidistantly spaced apart 90 degrees from the C-paddle 250, and markers 160A and 160B are disposed 180 degrees from one another on the capsule 150.

As can be seen in FIG. 5A, the aligned position is such that the commissures 209 of the transcatheter heart valve prosthesis 200 are aligned with the native commissures 120 of the idealized native aortic valve. Thus, the aligned position is a rotational orientation of the capsule 150. When viewed in a fluoroscopic image in the cusp overlap viewing angle COVA (“cusp overlap viewing angle image”), as shown in FIG. 5B, the markers 160A, 160B, will be seen as aligned with the marker 160A centered within the width of the marker 160B. As can be seen in FIG. 5A, the marker 160A is directly behind the marker 160 when viewed from the cusp overlap viewing angle COVA. Thus, in the 2-dimensional cusp overlap viewing angle image of FIG. 5B, the markers 160A, 160B are seen as aligned. Further, due to the markers 160A, 160B being longitudinally offset, the marker 160A is seen as above the marker 160B in the cusp overlap viewing angle image of FIG. 5B. The C-shape of the marker 160B also enables a clinician to confirm that the marker 160B is anterior to the marker 160A. If the C-shape of the marker 160B were backwards in the image of FIG. 5B, then the marker 160B would be posterior in the image of FIG. 5B.

Thus, when the capsule 150 of the delivery system is delivered to the native aortic valve, if the markers 160A, 160B are shown in the cusp overlap viewing angle image with the marker 160A centered above the marker 160B, the clinician knows that the capsule is properly rotationally oriented such that the commissures 209 of the transcatheter heart valve prosthesis 200 will not block the left and right coronary ostia LCO, ROC. Therefore, the clinician can proceed to retract the capsule 150 to deploy the transcatheter heart valve prosthesis 200.

As discussed above, the arrangement and size of the markers 160A, 160B on the capsule 150 enable a clinician to know whether the capsule 150, and hence the transcatheter valve prosthesis 200, is within the allowable angle of rotation θ (Theta) or tolerance angle with respect to the native aortic valve by visualizing the markers 160A, 160B in the cusp overlap viewing angle image, as explained in more detail below.

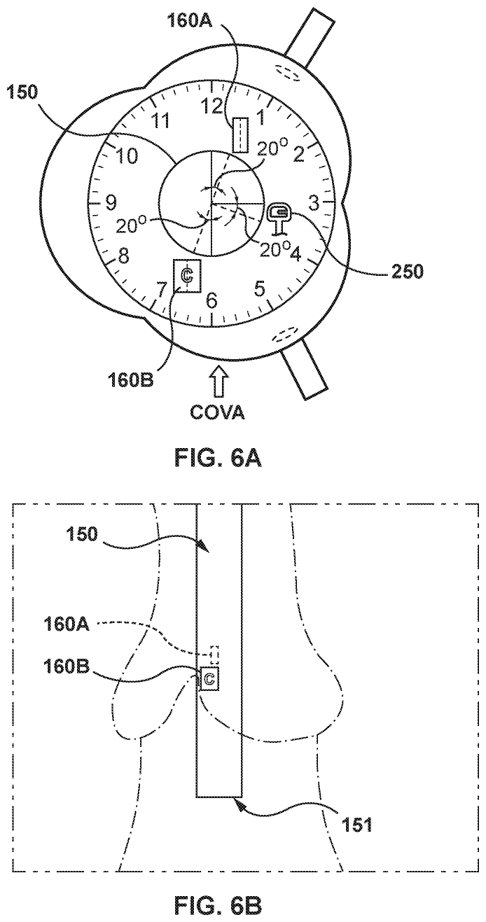

In particular, FIGS. 6A-6B show the capsule 150 disposed within the native aortic valve and rotated 20 degrees clockwise from the aligned position of FIGS. 5A-5B. This may also be referred to as a first position. As discussed above with respect to FIG. 4, in an embodiment, the commissures 209 of the transcatheter heart valve prosthesis 200 can be up to 20 degrees rotationally offset from the native commissures 120 and still be acceptably oriented. Thus, FIGS. 6A-6B show such a position.

FIG. 6A shows a schematic view of a native aortic valve as viewed from the aorta, with the capsule 150 including the markers 160A, 160B shown, and the C-paddle 250 of the transcatheter heart valve prosthesis 200 also shown. As can be seen in FIG. 6A, the capsule 150 is rotated 20 degrees clockwise from the aligned position of FIG. 5A. However, as can also be seen in FIG. 6A, C-paddle 250, and hence the commissure 209 aligned therewith, is spaced 40° from the left coronary ostium LCO, and is spaced 80 degrees from the right coronary ostium. As discussed above, this is an acceptable position.

When viewed in the 2-dimensional coronary overlap viewing angle image, as shown in FIG. 6B, this first position is shown as the marker 160A shifted laterally to the right edge of the marker 160B, but the right edge of the marker 160A still within the boundary of the width of the marker 160B, that is, within the right edge of the marker 160B. Further, due to the markers 160A, 160B being longitudinally offset, the marker 160A is seen as above the marker 160B in the cusp overlap viewing angle image of FIG. 6B. The C-shape of the marker 160B also enables a clinician to confirm that the marker 160B is anterior to the marker 160A. If the C-shape of the marker 160B were backwards in the image of FIG. 6B, then the marker 160B would be posterior in the image of FIG. 6B.

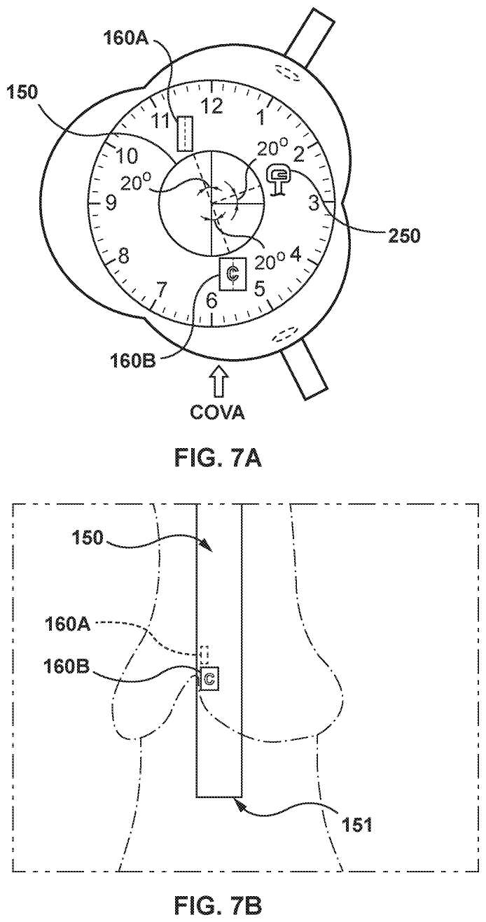

Similarly, FIGS. 7A-7B show the capsule 150 disposed within the native aortic valve and rotated 20 degrees counter-clockwise from the aligned position of FIGS. 5A-5B. This may also be referred to as a second position. As discussed above with respect to FIG. 4, in an embodiment, the commissures 209 of the transcatheter heart valve prosthesis 200 can be up to 20 degrees rotationally offset from the native commissures 120 and still be acceptably oriented. Thus, FIGS. 7A-7B show such a position.

FIG. 7A shows a schematic view of a native aortic valve as viewed from the aorta, with the capsule 150 including the markers 160A, 160B shown, and the C-paddle 250 of the transcatheter heart valve prosthesis 200 also shown. As can be seen in FIG. 7A, the capsule 150 is rotated 20 degrees counter-clockwise from the aligned position of FIG. 5A. However, as can also be seen in FIG. 7A, C-paddle 250, and hence the commissure 209 aligned therewith, is spaced 40° from the right coronary ostium RCO, and is spaced 80 degrees from the left coronary ostium LCO. As discussed above, this is an acceptable position.

When viewed in the 2-dimensional coronary overlap viewing angle image, as shown in FIG. 7B, this second position is shown as the marker 160A shifted laterally to the left edge of the marker 160B, but the left edge of the marker 160A still within the boundary of the width of the marker 160B, that is, within the left edge of the marker 160B. Further, due to the markers 160A, 160B being longitudinally offset, the marker 160A is seen as above the marker 160B in the cusp overlap viewing angle image of FIG. 7B. The C-shape of the marker 160B also enables a clinician to confirm that the marker 160B is anterior to the marker 160A. If the C-shape of the marker 160B were backwards in the image of FIG. 7B, then the marker 160B would be posterior in the image of FIG. 7B.

Thus, given the 20 degree tolerance or allowable angle of rotation θ (Theta) in either direction (clockwise and counter-clockwise), and the size of the capsule, the markers 160A, 160B may be sized such that in the cusp overlap view angle image, a clinician can determine if the capsule 150, and hence the transcatheter heart valve prosthesis 200 disposed therein, is within the allowable angle of rotation θ (Theta) or tolerance. In the example of the capsule 150 and markers 160A, 160B FIGS. 4A-4B, when viewed in the cusp overlap viewing angle image, a clinician can determine that the capsule 150 is in an allowable angle of rotation θ (Theta) if the marker 160A is disposed within the boundaries of the marker 160B, as described above.

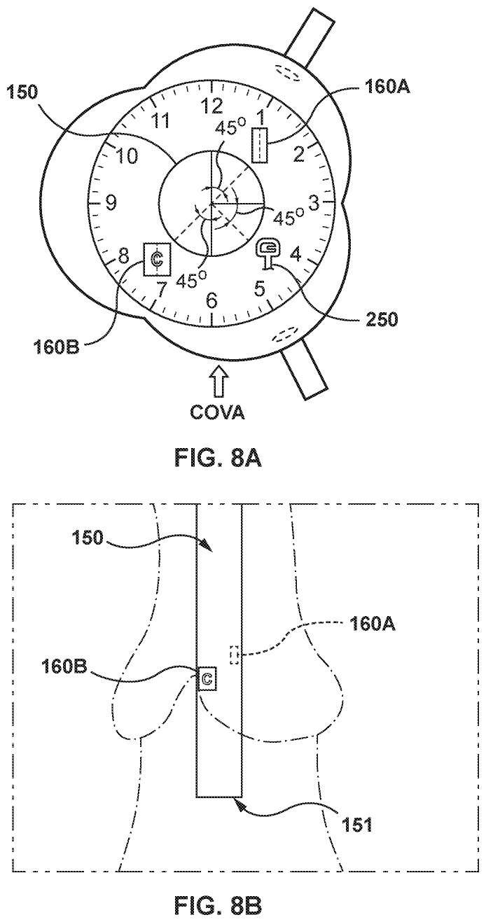

FIGS. 8A-8B show markers 160A, 160B of the capsule 150 and the C-paddle 250 of the transcatheter heart valve prosthesis 200 in an unaligned position. The unaligned position of the capsule 150 is defined as any position of the capsule 150 when the capsule 150 is rotated outside of the allowable angle of rotation θ (Theta). As described above, in an example, the allowable angle of rotation θ (Theta) is defined as a 20 degree rotational tolerance in each direction (clockwise and counterclockwise) from the aligned position of the capsule 150. In other words, the capsule 150 may be rotated a maximum of 20 degrees clockwise or counterclockwise from the aligned position to remain within the allowable angle of rotation θ (Theta). In other words, if the capsule 150 is rotated more than 20 degrees clockwise or counterclockwise from the aligned position, then the capsule 150 is in an unaligned position. Deploying the transcatheter heart valve prosthesis 200 when the capsule 150 is in an unaligned position would unacceptably increase the risk of the commissures 209 of the prosthesis 200 blocking at least one of the coronary ostia (LCO, RCO).

FIG. 8A shows the capsule 150 of the delivery system 100 within an exemplary native aortic valve as viewed from the aorta (in FIG. 8A from above) when the capsule 150 is in an unaligned position. As can be seen, the capsule 150 is rotated 45 degrees clockwise from the aligned position, for example, to show the capsule 150 in an unaligned position outside the allowable angle of rotation θ (Theta). In the unaligned position shown in FIG. 8A, the marker 106A, the marker 160B, and the C-paddle 250 of the transcatheter heart valve prosthesis 200 are also rotated 45 degrees clockwise from the aligned position with the capsule 150. In the unaligned position, as shown in FIG. 8A, the marker 160A is rotated 45 degrees clockwise from 12 o'clock, marker 160B is rotated 45 degrees clockwise from 6 o'clock, and the C-paddle 250 of the transcatheter heart valve prosthesis 200 is rotated 45 degrees clockwise from 3 o'clock. The unaligned position shown in FIGS. 8A-8B are exemplary in nature and not meant to be limiting. If the capsule 150 is rotated from the aligned position in any either direction more than the allowable angle of rotation θ (Theta) (20 degrees in the present example), the capsule 150 and the transcatheter heart valve prosthesis 200 are described as being in an unaligned position that is outside of the allowable angle of rotation θ (Theta).

As can be seen in FIG. 8A, in the idealized native aortic valve, capsule 150 is oriented 45 degrees from the aligned position in a clockwise direction, the C-paddle 250 of the transcatheter heart valve prosthesis 200, which is aligned with one of the commissures 209 of the transcatheter heart valve prosthesis 200, is disposed adjacent to the left coronary ostium LCO, thereby risking that the valve structure 204 of the transcatheter heart valve prosthesis 200 will block access to the left coronary ostium LCO. Given that another commissure 209 of the transcatheter heart valve prosthesis 200 is located 120° counterclockwise from the commissure 209 aligned with the C-paddle 250, such a commissure 209 would be located adjacent the right coronary ostium RCO, thereby risking that the valve structure 204 of the transcatheter heart valve prosthesis 200 will block access to the right coronary ostium RCO. Such a situation is undesirable.

When viewed in the 2-dimensional coronary overlap viewing angle image, as shown in FIG. 8B, the unaligned position shown in FIG. 8A appears as the marker 160A shifted laterally to the right and the marker 160B shifter laterally to the left. Importantly, as can be seen in FIG. 8B, the marker 160A is not within the left and right boundaries of the marker 160B. Therefore, a clinician viewing the cusp overlap viewing angle image shown in FIG. 8B can determine that the capsule 150 is in an unaligned rotation position with respect to the native heart valve. In such a position, as explained above, it is not desirable to deploy the transcatheter heart valve prosthesis 200. Therefore, if the capsule 150 is in an unaligned position, then the delivery system 100, or at least a portion thereof including the capsule 150, may be rotated such that the capsule is in the aligned position or within the allowable angle of rotation.

Thus, the embodiment of FIGS. 4A-4B and the method described with respect to FIGS. 5A-8B enable a clinician to determine if the capsule 150 is in an acceptable rotational position determining whether the capsule 150 is within the allowable angle of rotation from the aligned position. In the embodiment of the capsule 150 with the markers 160A, 160B as described, in the cusp overlap viewing angle image, if the marker 160A is between the left and right edges of the marker 160B, then capsule 150 is within the allowable rotational orientation within the native heart valve such that the valve structure 204 blocking the left and/or right coronary ostia LCO, RCO is unlikely. Thus, the capsule 150 may be retracted to enable the transcatheter heart valve prosthesis 200 disposed therein to be released and deployed within the native heart valve. If, on the other hand, the marker 160A is not disposed between the left and right edges of the marker 160B, then the capsule 150 is outside of the allowable rotational orientation within the native heart valve such that there is a risk that the valve structure 204 would block the left and/or right coronary ostia LCO, RCO. In such a situation, the capsule 150 may be rotated until the marker 160A is between the left and right edges of the marker 160B.

Those skilled in the art would recognize that the embodiment of FIGS. 4A-4B is merely one example, but that the concept described with respect to FIGS. 4A-4B may be utilized in other examples. For example, and not by way of limitation, the sizes of the markers 160A, 160B may change with changes to the size of the capsule 150 and/or a change to the allowable angle of rotation. However, the concept does not change that the markers 160A, 160B are sized and located on the capsule 150 such that when viewed in the cusp overlap viewing angle image, if the marker 160A is between the left and right edges of the marker 160B, then the capsule 150 is sufficiently rotationally aligned such that the transcatheter heart valve prosthesis 200 may be deployed from the capsule 150. If, on the other hand, the marker 160A is not between the left and right edges of the marker 160B when viewed in the cusp overlap viewing angle image, then the capsule 150 is not sufficiently rotationally aligned within the native heart valve. In such a situation the capsule 150 can be rotated until the marker 160A is between the left and right edges of the marker 160B, and then the transcatheter heart valve prosthesis 200 can be deployed therefrom.

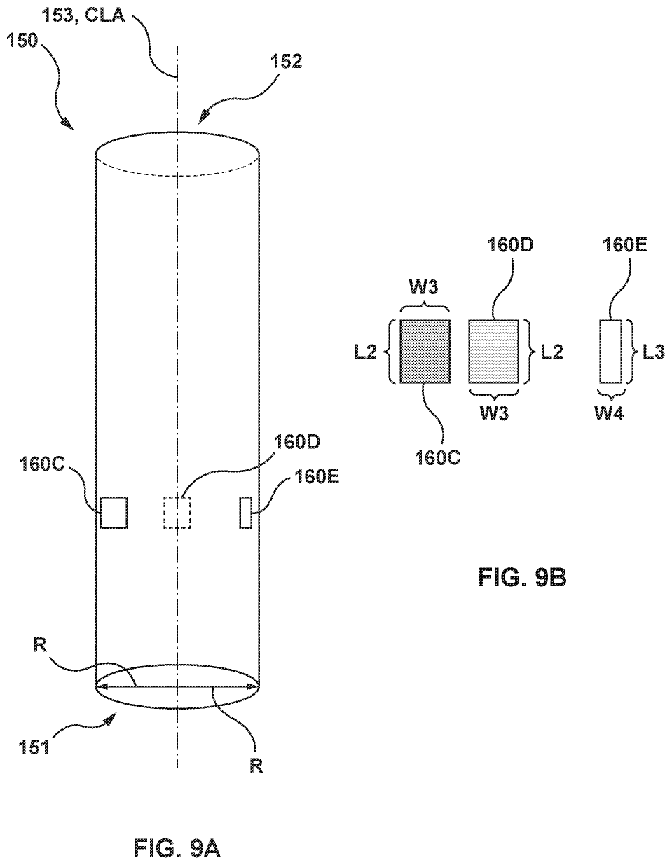

FIGS. 9A-9B show another embodiment of imaging markers on the capsule 150 of the delivery system 100. FIG. 9A shows a close up view of the capsule 150 of the delivery system 100. As can be seen, the capsule 150 of the delivery system 100 is a tubular structure including a distal end 151 and a proximal end 152. An interior surface of the capsule 150 defines a central lumen 153 that runs parallel to the central longitudinal axis CLA of the delivery system 100 and extends an entire longitudinal length of the capsule 150, extending from the distal end 151 to the proximal end 152. In an embodiment, the capsule 150 has a radius R of about 2.5-3.0 mm. However, this is not meant to be limiting, and the capsule 150 may have a radius (and diameter) as required to radially constrain a given transcatheter heart valve prosthesis. Thus, for transcatheter heart valve prostheses with a larger diameter when radially compressed, the capsule may have a larger diameter, and for transcatheter heart valve prostheses with a smaller diameter when radially compressed, the capsule may have a smaller diameter. The capsule 150, disposed at the distal end 103 of the delivery system 100, constrains the transcatheter heart valve prosthesis 200 in the radially compressed configuration, shown in FIG. 2B, during delivery to the treatment site. When the delivery system 100 is disposed at the treatment site, in this example a native aortic valve, the capsule 150 is withdrawn proximally (in FIG. 9A upwardly) to expose the self-expanding transcatheter heart valve prosthesis 200.

As shown in FIG. 9A, the capsule 150 includes imaging markers 160 disposed on the capsule 150. More specifically, the imaging markers 160 can be attached to, positioned in, and/or formed in the capsule 150 utilizing any type of processes and/or procedure. In any embodiment, the imaging markers 160 may include radiopaque or other material that allows the marker 160 to be detected and/or viewed under radiography during the implantation of the transcatheter heart valve prosthesis 200 via the delivery system 100. Examples of radiopaque materials include metals, e.g., platinum-iridium, gold, iridium, palladium, rhodium, titanium, tantalum, tungsten and alloys thereof. Other examples of radiopaque material include polymeric materials, e.g., nylon, polyurethane, silicone, PEBAX, PET, polyethylene, that have been mixed or compounded with compounds of barium, bismuth and/or zirconium, e.g., barium sulfate, zirconium oxide, bismuth sub-carbonate, etc. In embodiments, gold is a preferred marker material due to its visibility. Further, in addition to the radiopaque materials noted above, the imaging markers 160 can be a feature on the capsule 150 of the delivery system 100 that can be seen under fluoroscopy as distinguished from other features of the delivery system 100.

In the embodiment of FIGS. 9A-9B, the capsule 150 includes three markers 160C, 160D, 160E. As shown in FIG. 9A, the each of the markers 160C, 160D, 160E are disposed 120° from the other two markers. In other words, the markers 160C, 160D, 160E are evenly spaced around the circumference of the capsule 150. As can be seen in FIG. 9A, the marker 160D in that particular view is in the “back” of the capsule. With the markers 160C and 160E each 120 degrees apart from the marker 160D in opposite directions, the markers 160C and 160E may be described as being on the “side” of the capsule 150, but as shown, would also be on the “front” half of the capsule 105. In a 2-dimensional view, the markers 160C and 160E would appear to left and right sides of the capsule 150. Those skilled in the art will realize that “front” and “back” are used with reference only to FIG. 9A to refer to the relative positions of the markers 160C, 160D, 160E in that view, and are not meant to be limiting.

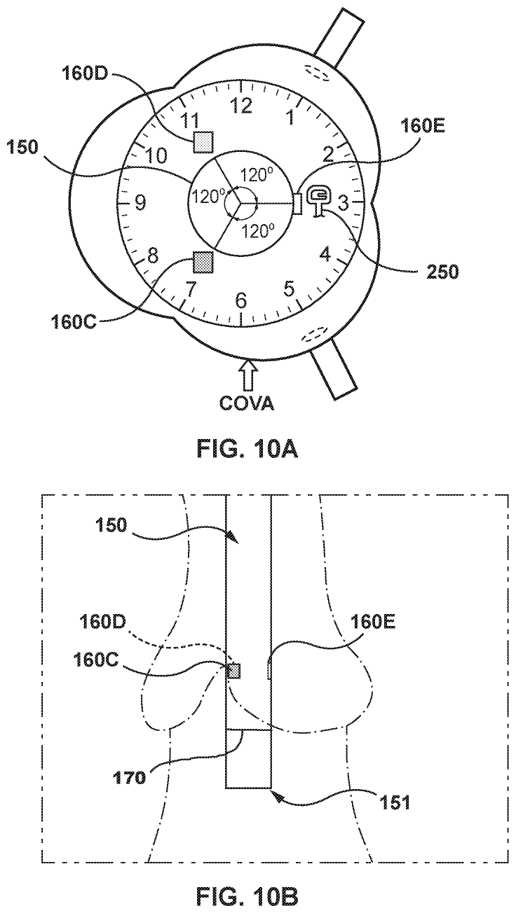

Further, in the embodiment of FIGS. 9A-9B, the markers 160C, 160D, 160E are aligned with respect to a circumferential line drawn around the circumference of the capsule 150. In other words, the markers 160C, 160D, 160E are disposed at the same longitudinal location on the capsule. In other words, a longitudinal distance from the proximal end 152 of the capsule 150 to each marker is the same, as is a longitudinal distance from the distal end 151 of the capsule to each marker. However, this is not meant to be limiting, and one or more of the markers may be longitudinally offset from the other markers. Further, the longitudinal distance of the markers 160C, 160D, 160E may be vary. Thus, the longitudinal locations shown in FIGS. 9A, 10B, 11B, 12B, and 13B are not meant to be limiting. For example, and not by way of limitation, the distance from the distal end of the capsule can be more or less than shown in FIGS. 10B, 11B, 12B, and 13B. As can be seen in FIGS. FIGS. 10B, 11B, 12B, and 13B, the capsule 150 may also include a depth marker 170. In an embodiment, the markers 160C, 160D, 160E may be aligned with the depth marker 170, or may be placed at the located of the depth marker 170 to replace the depth marker 170. In such an embodiment, the markers 160C, 160D, 160E may be used both for depth and for rotational orientation, as described below.

FIG. 9B shows a close up view of markers 160C, 160D, 160E. In the embodiment of FIGS. 9A-9B, each of the markers 160C, 160D, 160E are rectangular in shape. However, this is not meant to be limiting, and other shapes may be used. The markers 160C and 160D are sized such that when viewed in a fluoroscopic image in a cusp overlap view, the position of the marker 160C and 160D relative to each other enables a clinician to determine whether the transcatheter heart valve prosthesis 200 is rotationally oriented such as to avoid blocking the ostia of the coronary arteries, as described in more detail below. Thus, the markers 160C, 160D are sized relative to each other to provide a “tolerance” of positions relative to each other when viewed in a fluoroscopic image, as described in more detail below. In the embodiment of FIGS. 9A-9B, and as described in more detail below, when the markers 160C and 160D overlap each other, or their width edges are touching, the capsule 150 is rotationally oriented such that the commissure 209 of the heart valve prosthesis 200 is within the 40 degrees tolerance from the native right-left commissure 120B. Thus, in an example embodiment with a capsule diameter of about 5.56 mm (radius R of about 2.78 mm), and given the allowable angle of rotation O or tolerance angle described above of 40 degrees (about 0.7 radians) (20 degrees in either direction), each of the markers 160C, 160D has a width W3 of about .97 mm (W3 =allowable angle of rotation (about 0.7 radian)×capsule radius (2.78 mm)×0.5). The marker 160E may be the same size and the markers 160C, 160D, or may be a different size. Although a specific example is given above with respect to the size of the markers, this is not meant to me limiting, and the size may vary depending on the size of the capsule, the allowable tolerance angle, and other factors.

In an embodiment the, marker 160E has a width of in the range of 1.0-1.5 mm, although this is not mean to be limiting. The markers 160, 160d, 160E may all have the same length L2, L3, but this is not meant to be limiting. For example, as described in more detail below, in the aligned position, the marker 160C obscures the marker 160D in the cusp overlap viewing angle image. However, in other embodiments, it may be desirable for the marker 160D to be longer than the marker 160C so that it is clear that the two markers are aligned in the image. In an example, the lengths L2, L3 may be in the range of 1 to 8 mm, but this is not mean to be limiting. Although not shown in this embodiment, one of the markers 160C, 160D could include an asymmetrical shape, such as the C-shape described above with respect to FIGS. 4A-4B, to assist in determining whether that marker is anterior or posterior in the fluoroscopic image with the gantry in the cusp overlap viewing angle (also referred to as the “cusp overlap viewing angle image”). Those skilled in the art would recognize that other asymmetric shapes may be utilized to assist in determining the orientation of the capsule 150 during implantation.

In the embodiment shown, the marker 160E is aligned with the C-paddle 250 of the transcatheter heart valve prosthesis 200, which is axially aligned with one of the commissures 209 of the valve structure 204. With the markers 160C, 160D each 120 degrees from the marker 160E (in opposite directions), the markers 160C, 160D are also each axially aligned with one of the commissures 209 of the valve structure 204. However, this is not meant to be limiting, and any of the markers 160C, 160D, 160C may be aligned with the C-paddle 250.

With the above understanding of the markers 160C, 160D, 160E on the capsule 150 of the delivery system 100, a system and method of rotationally aligning the capsule 150 of the delivery system 100, and therefore the transcatheter heart valve prosthesis 200, will now be described. As known to those skilled in the art, the transcatheter heart valve prosthesis 200 may be delivered percutaneously via femoral access. In particular, in the example of a self-expanding transcatheter heart valve prosthesis, e.g. the transcatheter heart valve prosthesis 200, the prosthesis is constrained in a radially compressed configuration by, for example, the capsule 150 of the delivery system 100. Characteristics of a patient's native anatomy may be determined prior to the procedure, such as by a CT scan. In particular, the coronary ostia (LCO, RCO) may be located using a CT scan. Using this planning CT, a determination may be made prior to the procedure regarding orientation of the delivery system, and hence the transcatheter heart valve prosthesis, when delivering the transcatheter heart valve prosthesis.

For example, and not by way of limitation, the delivery system 100 can be arranged such that the flush port 116 is aligned with the C-paddle 250 of the transcatheter heart valve prosthesis 200, which is aligned with one of the commissures 209 of the valve structure 204. Using pre-procedure CT, the orientation of a feature of the delivery system, such as the flush portion 116, that has a known relationship to a feature of the transcatheter heart valve prosthesis, such as one of the commissures 209 of the transcatheter heart valve prosthesis 200, may be further defined by the specific patient anatomy. Thus, using pre-procedure planning, a prediction can be made regarding a preferred orientation of the delivery system, such as the delivery system 100, to reduce coronary artery ostia overlap.

Further, during the procedure, the coronary overlap view and the markers 160C, 160D, 160E may be used to confirm that the transcatheter heart valve prosthesis is rotationally aligned such as to not cause coronary obstruction. The capsule 150 may then be retracted proximally to expose the transcatheter heart valve prosthesis 200, enabling the transcatheter heart valve prosthesis 200 to self-expand.

Thus, when the delivery system 100 is advanced to the site of a native heart valve, for example a native aortic valve, the markers 160C, 160D, 16E can be used to confirm that the capsule 150 is in a desired rotational orientation within the native valve. In particular, the cusp overlap viewing angle discussed above may be utilized to determine if the capsule 150, and hence the transcatheter heart valve prosthesis 200 disposed therein, are properly rotationally oriented.

FIGS. 6A-6B depict the markers 160C, 160D, 160E on the capsule 150 of the delivery system 100, as introduced in FIGS. 9A-9B, in a specific rotational position termed an “aligned position”, according to embodiments hereof. The “aligned position” as used herein is with respect to the capsule's 150 rotational orientation with respect to the native aortic valve, and hence the position of the transcatheter heart valve prosthesis 200 disposed within the capsule 150 with respect to the native aortic valve.

As stated previously, markers 160C, 160D, 160E are disposed 120 degrees from each other on the capsule 150. The transcatheter heart valve prosthesis 200 is disposed within the central lumen 153 of the capsule 150 in the compressed configuration. In the embodiment described, when the transcatheter heart valve prosthesis 200 is loaded into the capsule 150 in the compressed configuration, the C-paddle 250 of the transcatheter heart valve prosthesis 200 is aligned with the marker 160E, as best shown in FIG. 10A. FIG. 10A shows the capsule 150 of the delivery system 100 within an exemplary native aortic valve as viewed from the aorta. The capsule 150 includes the markers 160C, 160D, 160E from FIGS. 9A-9B. As can be seen, each marker 160C, 160D, 160E is aligned with one of the native commissures 120A, 120B, 120C. With reference to the clock face, the marker 160C is positioned at 7 o'clock, the marker 160D is positioned at 11 o'clock, and the marker 160E is position at 3 o'clock. The C-paddle 250 is also disposed at 3 o'clock.

In the idealized native aortic valve shown, this is the aligned position as each of the commissures 209 is aligned with a respective one of the native commissures 120A, 120B, 120C. Thus, the aligned position is a rotational orientation of the capsule 150 with respect to the native aortic valve. When viewed in a fluoroscopic image in the cusp overlap viewing angle COVA (“cusp overlap viewing angle image”), as shown in FIG. 10B, the markers 160C, 160D will be seen as aligned such that the marker 160C, which is in the “front” based on the viewing angle, completely obscures the marker 160D, which is in the “back” based on the viewing angle. In the 2-dimensional cusp overlap viewing angle image, the markers 160C, 160D are aligned with each towards the left side of the capsule 150 and the marker 160E is disposed towards the right side of the capsule 150. While the markers 160C, 160D have been described as having the same length L2 (appearing as a height in FIG. 10B), the marker 160D could be longer than the marker 160C such that it is clear that they are aligned. Further, although not shown in this embodiment, the marker 160C could include a C-shape, as described above with respect to the marker 160B, to assist in confirming that the marker 160C is anterior to the marker 160D, as explained above.

Thus, when the capsule 150 of the delivery system is delivered to the native aortic valve, if the markers 160C, 160D are shown in the cusp overlap viewing angle image with the markers 160C, 160D aligned and to the left side of the capsule 150, and the marker 160E is shown towards the right side of the capsule 150, then the capsule is properly rotationally oriented such that the commissures 209 of the transcatheter heart valve prosthesis 200 will not block the left and right coronary ostia LCO, ROC. Therefore, the capsule 150 may be retracted to deploy the transcatheter heart valve prosthesis 200. Further, the marker 160E may be excluded.

As discussed above, the arrangement and size of the markers 160C and 160D on the capsule 150 enable a clinician to know whether the capsule 150, and hence the transcatheter valve prosthesis 200, is within a desired tolerance of rotational orientation with respect to the native aortic valve by visualizing the markers 160C, 160D in the cusp overlap viewing angle image, as explained in more detail below.

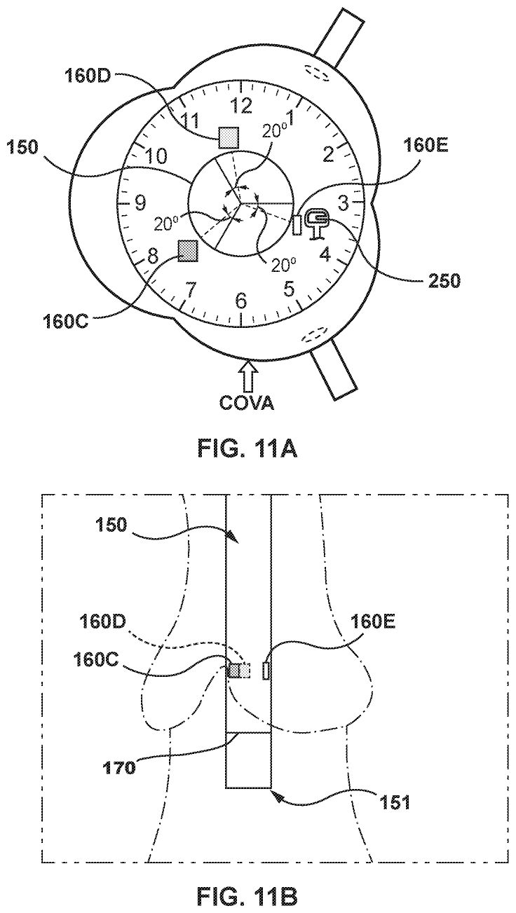

In particular, FIGS. 11A-11B show the capsule 150 disposed within the native aortic valve and rotated 20 degrees clockwise from the aligned position of FIGS. 10A-10B. This may also be referred to as a first position. As discussed above with respect to FIG. 4, in an embodiment, the commissures 209 of the transcatheter heart valve prosthesis 200 can be up to 20 degrees rotationally offset from the native commissures 120 and still be acceptably oriented. Thus, FIGS. 11A-11B show such a position.

FIG. 11A shows a schematic view of a native aortic valve as viewed from the aorta, with the capsule 150 including the markers 160C, 160D, 160E shown, and the C-paddle 250 of the transcatheter heart valve prosthesis 200 also shown. As can be seen in FIG. 11A, the capsule 150 is rotated 20 degrees clockwise from the aligned position of FIG. 10A. However, as can also be seen in FIG. 11A, C-paddle 250, and hence the commissure 209 aligned therewith, is spaced 40 degrees from the left coronary ostium LCO, and is spaced 80 degrees from the right coronary ostium. As discussed above, this is an acceptable position.

When viewed in the 2-dimensional coronary overlap viewing angle image, as shown in FIG. 11B, this first position is shown as the marker 160C shifted laterally to the left and the marker 160D shifted laterally to the left such that the right edge of the marker 160C is aligned with the left edge of the marker 160D. In other words, the markers 160C and 160D are side-by-side (with the marker 160C to the left) with no gap between the markers 160C, 160D. The marker 160E remains towards the right of the capsule 150 in the cusp overlap viewing angle image of the first position. As discussed above, the marker 160 may further include a C-shape to confirm that the marker 160C is anterior to the marker 160D.

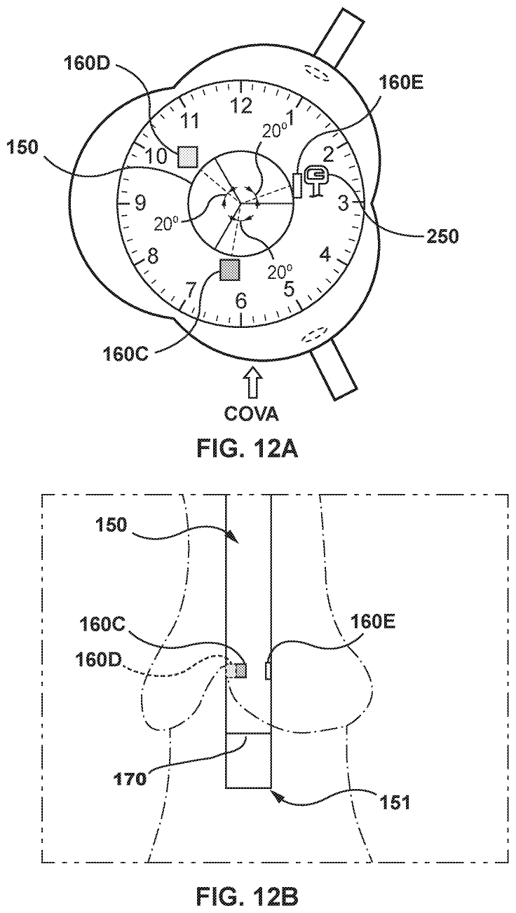

Similarly, FIGS. 12A-12B show the capsule 150 disposed within the native aortic valve and rotated 20 degrees counter-clockwise from the aligned position of FIGS. 10A-10B. This may also be referred to as a second position. As discussed above with respect to FIG. 4, in an embodiment, the commissures 209 of the transcatheter heart valve prosthesis 200 can be up to 20 degrees rotationally offset from the native commissures 120 and still be acceptably oriented. Thus, FIGS. 12A-12B show such a position.

FIG. 12A shows a schematic view of a native aortic valve as viewed from the aorta, with the capsule 150 including the markers 160C, 160D, 160E shown, and the C-paddle 250 of the transcatheter heart valve prosthesis 200 also shown. As can be seen in FIG. 11A, the capsule 150 is rotated 20 degrees counterclockwise from the aligned position of FIG. 10A. However, as can also be seen in FIG. 12A, C-paddle 250, and hence the commissure 209 aligned therewith, is spaced 40° from the right coronary ostium RCO, and is spaced 80 degrees from the left coronary ostium. As discussed above, this is an acceptable position.

When viewed in the 2-dimensional coronary overlap viewing angle image, as shown in FIG. 12B, this second position is shown as the marker 160C shifted laterally to the right and the marker 160D shifted laterally to the left such that the left edge of the marker 160C is aligned with the right edge of the marker 160D. In other words, the markers 160C and 160D are side-by-side (with the marker 160D to the left) with no gap between the markers 160C, 160D. The marker 160E remains towards the right of the capsule 150 in the cusp overlap viewing angle image of the second position. As discussed above, the marker 160 may further include a C-shape to confirm that the marker 160C is anterior to the marker 160D.

Thus, given the 20 degree tolerance in either direction (clockwise and counter-clockwise), and the size of the capsule, the markers 160C, 160D may be sized such that in the cusp overlap view angle image, a clinician can determine if the capsule 150, and hence the transcatheter heart valve prosthesis 200 disposed therein, is within the acceptable tolerance of rotational orientation. In the example of the capsule 150 and markers 160C, 160D of FIGS. 9A-9B, when viewed in the cusp overlap viewing angle image, a clinician can determine that the capsule 150 is in an acceptable rotational orientation if the markers 160C 160D are disposed at least partially overlapped or side-by-side with no gap between them, and the marker 1600E is towards the right of the capsule 150. Thus, the sizes and arrangement of the markers 160C, 160D provides a simple indicator of whether the capsule 150 is rotationally oriented within the acceptable angle of rotation.

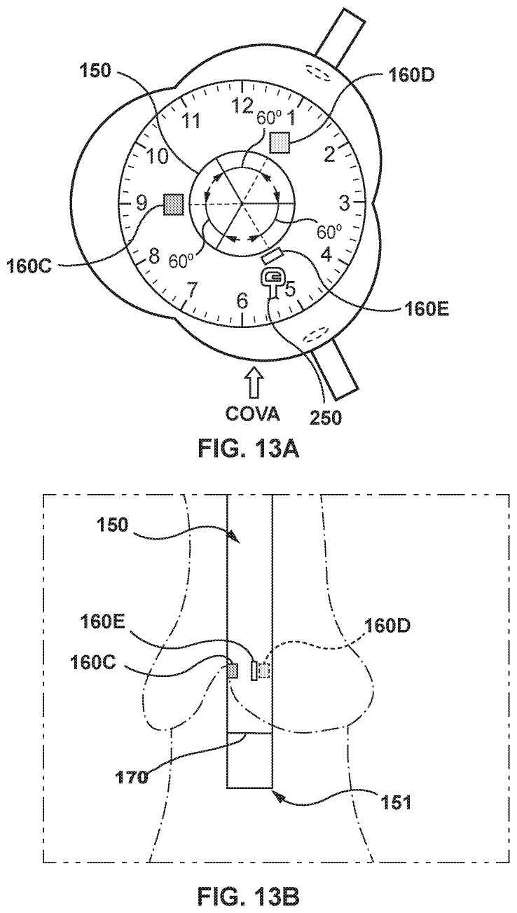

FIGS. 13A-13B show the markers 160C, 160D, 160E of the capsule 150 and the C-paddle 250 of the transcatheter heart valve prosthesis 200 in an unaligned position. The unaligned position of the capsule 150 is defined as any position of the capsule 150 when the capsule 150 is rotated outside of the allowable angle of rotation θ (Theta). As described above, in an example, the allowable angle of rotation θ (Theta) is defined as a 20 degree rotational tolerance in each direction (clockwise and counterclockwise) from the aligned position of the capsule 150. In other words, the capsule 150 may be rotated a maximum of 20 degrees clockwise or counterclockwise from the aligned position to remain within the allowable angle of rotation θ (Theta). In other words, if the capsule 150 is rotated more than 20 degrees clockwise or counterclockwise from the aligned position, then the capsule 150 is in an unaligned position. Deploying the transcatheter heart valve prosthesis 200 when the capsule 150 is in an unaligned position would unacceptably increase the risk of the commissures 209 of the prosthesis 200 blocking at least one of the coronary ostia (LCO, RCO).

FIG. 13A shows the capsule 150 of the delivery system 100 within an exemplary native aortic valve as viewed from the aorta (in FIG. 13A from above) when the capsule 150 is in an unaligned position. As can be seen, the capsule 150 is rotated 45 degrees clockwise from the aligned position, for example, to show the capsule 150 in an unaligned position outside the allowable angle of rotation θ (Theta). In the unaligned position shown in FIG. 13A, the marker 106C, the marker 160C, the marker 160E, and the C-paddle 250 of the transcatheter heart valve prosthesis 200 are also rotated 45 degrees clockwise from the aligned position with the capsule 150. In the unaligned position, as shown in FIG. 13A, the marker 160C is rotated 45 degrees clockwise from 7 o'clock, marker 160D is rotated 45 degrees clockwise from 11 o'clock, and marker 160E and the C-paddle 250 of the transcatheter heart valve prosthesis 200 are rotated 45 degrees clockwise from 3 o'clock. The unaligned position shown in FIGS. 13A-13B are exemplary in nature and not meant to be limiting. If the capsule 150 is rotated from the aligned position in any either direction more than the allowable angle of rotation θ (Theta) (20 degrees in the present example), the capsule 150 and the transcatheter heart valve prosthesis 200 are described as being in an unaligned position that is outside of the allowable angle of rotation θ (Theta).

As can be seen in FIG. 13A, in the idealized native aortic valve, capsule 150 is oriented 45 degrees from the aligned position in a clockwise direction, the C-paddle 250 of the transcatheter heart valve prosthesis 200, which is aligned with one of the commissures 209 of the transcatheter heart valve prosthesis 200, is disposed adjacent to the left coronary ostium LCO, thereby risking that the valve structure 204 of the transcatheter heart valve prosthesis 200 will block access to the left coronary ostium LCO. Similarly, given that another commissure 209 of the transcatheter heart valve prosthesis 200 is located 120 degrees counterclockwise from the commissure 209 aligned with the C-paddle 250, such a commissure 209 would be located adjacent the right coronary ostium RCO, thereby risking that the valve structure 204 of the transcatheter heart valve prosthesis 200 will block access to the right coronary ostium RCO. Such a situation is undesirable.

When viewed in the 2-dimensional coronary overlap viewing angle image, as shown in FIG. 13B, the unaligned position shown in FIG. 13A appears as the marker 160C towards the left of the capsule 150, the marker 160D towards the right of the capsule 150, and the marker 160E between the markers 160C, 160D. As discussed above, because the markers 160C, 160D are not either overlapping or side-by-side with no gap therebetween (edge-to-edge), a clinician viewing the cusp overlap viewing angle image shown in FIG. 13B can determine that the capsule 150 is in an unaligned rotation position with respect to the native heart valve. In such a position, as explained above, it is not desirable to deploy the transcatheter heart valve prosthesis 200. Therefore, if the capsule 150 is in an unaligned position, then the delivery system 100, or at least a portion thereof including the capsule 150, may be rotated such that the capsule 150 is in the aligned position or within the allowable angle of rotation. However, those skilled in the art will recognize that the unaligned position of FIGS. 13A-13B is merely one example of an unaligned position, and other unaligned positions would appear in the cusp overlap viewing angle image as the markers 160C, 160C laterally spaced with a gap therebetween. such that not overlapping or not side-by-side with no gape

Thus, the embodiment of FIGS. 9A-9B and the method described with respect to FIGS. 10A-13B enable a clinician to determine if the capsule 150 is in an acceptable rotational position determining whether the capsule 150 is within the allowable angle of rotation from the aligned position. In the embodiment of the capsule 150 with the markers 160C, 160D, 160E as described, in the cusp overlap viewing angle image, if the markers 160C, 160D are overlapping or side-by-side with no gap therebetween, and the marker 160E is to the right of the markers 160C, 160D, then capsule 150 is within the allowable rotational orientation within the native heart valve such that the valve structure 204 blocking the left and/or right coronary ostia LCO, RCO is unlikely. Thus, the capsule 150 may be retracted to enable the transcatheter heart valve prosthesis 200 disposed therein to be released and deployed within the native heart valve.

If, on the other hand, the markers 160C, 1160D are laterally spaced such that there is a gap between them, then the capsule 150 is outside of the allowable rotational orientation within the native heart valve such that there is a risk that the valve structure 204 would block the left and/or right coronary ostia LCO, RCO. In such a situation, the capsule 150 may be rotated until the markers 160C, 160D are overlapping or side-by-side with no gap therebetween. Those skilled in the art would recognize that the embodiment of FIGS. 9A-9B is merely one example, but that the concept described with respect to FIGS. 9A-9B may be utilized in other examples. For example, and not by way of limitation, the sizes of the markers 160C, 160D may change with changes to the size of the capsule 150 and/or a change to the allowable angle of rotation. However, the concept does not change that the markers 160C, 160D are sized and located on the capsule 150 such that when viewed in the cusp overlap viewing angle image, if the markers 160C, 160D overlap or side-by-side with not gap therebetween, then the capsule 150 is sufficiently rotationally aligned such that the transcatheter heart valve prosthesis 200 may be deployed from the capsule 150.

If, on the other hand, the markers 160C, 160D are laterally spaced from each other such that there is a gap therebetween viewed in the cusp overlap viewing angle image, then the capsule 150 is not sufficiently rotationally aligned within the native heart valve. In such a situation the capsule 150 can be rotated until the markers 160C, 160D overlap or are side-by-side with no gap therebetween, and then the transcatheter heart valve prosthesis 200 can be deployed therefrom.

It should be understood that various embodiments disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single device or component for purposes of clarity, it should be understood that the techniques of this disclosure may be performed by a combination of devices or components.

1. A delivery system comprising:
a capsule disposed at a distal end of the delivery system and configured to radially constrain a heart valve prosthesis in a radially compressed configuration for delivery to a treatment site; and
a plurality of markers disposed on the capsule, wherein the plurality of markers are sized, shaped, and located on the capsule such that when viewed in a cusp overlap viewing angle image, the markers indicate whether the capsule is in a desired rotational orientation.

2. The delivery system of claim 1, wherein the desired rotational orientation defines an aligned position or a position within an allowable angle of rotation of the aligned position.

3. The delivery system of claim 1, wherein the plurality of markers comprise a first marker and a second marker, wherein the first marker and the second marker are spaced 180 degrees apart around a circumference of the capsule.

4. The delivery system of claim 3, wherein the first marker has a first width and the second marker has a second width, wherein the first width is larger than the second width.

5. The delivery system of claim 4, wherein the capsule is in the aligned position or the position within an allowable angle of rotation of the aligned position when the second marker is between a right edge and a left edge of the first marker in the cusp overlap viewing angle image.

6. The delivery system of claim 3, wherein the first marker and the second marker are longitudinally offset from each other such that one of the first marker and the second marker is closer to a distal end of the capsule than the other of the first marker and the second marker.

7. The delivery system of claim 4, wherein the second width is the allowable angle of rotation in radians times the capsule radius plus the first width.

8. The delivery system of claim 1, wherein the plurality of markers comprises a first marker, a second marker, and a third marker spaced 120 degrees apart around a circumference of the capsule, wherein the first marker, second marker, and third marker are configured to be aligned with a commissure of the transcatheter heart valve prosthesis configured to be received in the capsule.

9. The delivery system of claim 8, wherein the capsule is in the aligned position or the position within an allowable angle of rotation of the aligned position when the first marker and the second marker overlap or are side-by-side with no gap therebetween in the cusp overlap viewing angle image, and the third marker is towards the right of the capsule in the cusp overlap viewing angle image.

10. The delivery system of claim 8, wherein the first marker and the second marker each have a width about equal to the allowable angle of rotation in radians times a radius of the capsule and divided in half.

11. A method for rotationally aligning a capsule of a delivery system containing a transcatheter heart valve prosthesis within a native heart valve, the method comprising:
percutaneously delivering the capsule with the transcatheter heart valve prosthesis contained therein to the native heart valve, wherein the capsule includes a plurality of markers;
obtaining a cusp overlap viewing angle image of the capsule within the native heart valve;
determining, based on the cusp overlap viewing angle image and the plurality of markers, whether the capsule is in a desired rotational orientation; and
if the cusp overlap viewing angle image indicates that the capsule is not in the desired rotational orientation, rotating the capsule until the capsule is in the desired rotational orientation.

12. The method of claim 11, wherein the desired rotational orientation comprises an aligned position or a position within an allowable angle of rotation of the aligned position.

13. The method of claim 11 wherein the plurality of markers comprise a first marker and a second marker, wherein the first marker and the second marker are spaced 180 degrees apart around a circumference of the capsule, and a commissure of the transcatheter heart valve prosthesis is spaced 90 degrees from each of the first marker and the second marker.

14. The method of claim 13, wherein the first marker has a first width and the second marker has a second width, wherein the first width is larger than the second width.

15. The method of claim 14, wherein the determining whether the capsule is in a desired rotational orientation comprises determining, based on the cusp overlap viewing angle image, whether the second marker is between a right edge and a left edge of the first marker.

16. The method of claim 13, wherein the first marker and the second marker are longitudinally offset from each other such that one of the first marker and the second marker is closer to a distal end of the capsule than the other of the first marker and the second marker.

17. The method of claim 14, wherein the second width is the allowable angle of rotation in radians times the capsule radius plus the first width.

18. The method of claim 11, wherein the plurality of markers comprises a first marker, a second marker, and a third marker spaced 120 degrees apart around a circumference of the capsule, and wherein each of the first marker, second marker, and third marker is rotationally aligned with a corresponding commissure of the transcatheter heart valve prosthesis.

19. The method of claim 18, wherein determining whether the capsule is in the desired rotational orientation comprises determining, based on the cusp overlap viewing angle image, whether the first marker and the second marker overlap or are side-by-side with no gap therebetween and whether the third marker is towards the right of the capsule.

20. The method of claim 18, wherein the first marker and the second marker each have a width about equal to the allowable angle of rotation in radians times a radius of the capsule and divided in half.
